(19) United States
(12) Patent Application Publication
(10) Pub. No.: US 2020/0383449 A1
(43) Pub. Date: Dec. 10, 2020

(54) HAIR BAND WITH INTERCHANGEABLE ATTACHMENTS

(71) Applicants: Karolina WILCZYNSKA, Cambridge (CA); Natalia LEWANDOWSKA, Cambridge (CA)

(72) Inventors: Karolina WILCZYNSKA, Cambridge (CA); Natalia LEWANDOWSKA, Cambridge (CA)

(21) Appl. No.: 16/434,739

(22) Filed: Jun. 7, 2019

(51) Int. Cl. A45D 8/34; A61N 2/06

(52) U.S. Cl. CPC A45D 8/34; A45D 2008/006; A61N 2/06

(57) ABSTRACT

The present invention is a hair band providing a method to attach various accessories thereon to meet the needs of a large group of customers comprising a rectangular fabric strip folded and the longitudinal edges of the fabric strip are stitched together, resulting a fabric tube and the ends of the tube are stitched together to constitute a tubular annulus. An elastic band is extended inside the tube through a plurality of slits symmetrically provided adjacent to each other on longitudinal edge of the tube. This arrangement results to provide a fabric tube in which a portion of the elastic band is exposed through the slits of the fabric tube, so that the user can easily access to the exposed portion of the elastic band. The elastic band is secured on the tube by fasteners and can receive various accessories.

### FIELD OF THE INVENTION

The present invention relates in general to wearing accessories and in specific to decorative scrunchies with multiple use as a hair band or a wrist band.

### BACKGROUND OF THE INVENTION

Elastic bands with surrounding fabric referred to as “scrunchies” are often used as a pony-tail holder while providing fashion to one's overall appearance. The scrunchies existing in the market have various decorations. A traditional scrunchie can be placed on a ponytail by pulling the ponytail through the scrunchie, twisting the scrunchie then pulling the ponytail through the created loop. This process can be repeated until the ponytail is securely fastened. Usually in this process the decorative accessories, which are usually adhered or sewed on the fabric will become loose or detached. Also, most of the decorative accessories will be hidden in between the ruffles.

Usually the decorations comprise of fabric flowers, gems or other decorative accessories, which are attached by various attaching means on the fabric. The bunching of the fabric at the cross-over is so severe that causes the decorative accessories to be impaired or detached or especially if the scrunchie is made of delicate fabric may result to tear the fabric. So, a need exists for a scrunchie having the possibility to attach decorative accessories including charms, jewels, fabric decorations in a sturdy manner to prevent any detachment possibility.

### SUMMARY OF THE INVENTION

The present invention relates to improvements in a currently existing ponytail holders commonly called a scrunchie. The new scrunchie allows the user to change the ruffle distribution on the scrunchie. The new scrunchie also provides means to attach various accessories on the scrunchie. Users can customize their scrunchie with decorative accessories, which can be used as a hobby to trade and collect as a collection like pandora charms. The novel elements of the present scrunchies also function as therapeutic bracelet whenever it is put on a wrist.

Scrunchies, generally, comprise of a rectangular fabric strip that is folded, and the longitudinal edges of the fabric strip are stitched together. This forms a fabric tube. The ends of this fabric tube are stitched together to form a tubular annulus with a length of preferably from about 20 to 24 inches and a width from about 2 to 5 inches. An elastic band is then slidably inserted in the fabric tube and its two ends are attached together. The elastic band makes the fabric tube to become ruffled, thus forming the scrunchie. The tube may be made of various fabric materials. The prior art scrunchies do not allow the user to change the ruffle distribution since the elastic band is inside the tube and the user cannot change its position with respect to the fabric tube.

In accordance with the first embodiment of the present invention, a plurality of slits or cut-outs are provided adjacent to each other along on the surface of the fabric and along the length of the tube. The slit spacings are such that every two slits are close to each other and farther from the next two slits. The elastic band is then passed in and out of the two closely spaced slits, such that a small portion of the elastic band is exposed through the slits of the fabric tube. This allows users to easily access the exposed portion of the elastic band. Once the elastic band is inserted, then the fabric is folded and sewed (the slits are designed on the fabric prior to sewing the longitudinal edges of the fabric strip together and making a fabric tube). The elastic band is moved between adjacent slits so that the fabric is gathered into ruffles thereby causing the tube to create its final scrunchie shape. The user can move the fabric along the elastic band to form any desired ruffled distributions.

The slits can be made in various arrangements, such as on one side or on both sides of the tube. As the fabric is not twisted, one side of the fabric that is oriented outwardly becomes the display side, and the second side of the fabric faces the user's head.

In one embodiment of the same scrunchie, a plurality of attaching means are attached on the section of the elastic band that sticks out of the slits. The attaching means are various fasteners to receive decorative accessories on the fabric. This allows the users to attach accessories to the scrunchie.

In another embodiment of the present invention, a plurality of magnets is attached to the exposed sections of the elastic band. The scrunchie with magnets attachments is used as a therapeutic wristband. Suitable therapeutic magnets are attached to the fastener. The user can detach the scrunchie from his/her hair and dispose it around his/her wrist. By wearing the scrunchie around the wrist and position the magnets to face the wrist or forearm, the scrunchie becomes a therapeutic wrist band, while the decorative accessories are attached on the outer portion of the scrunchie.

The present invention can be produced as a kit with various accessories with various attachments, such as clips, pins, magnets or Velcro, with corresponding accessories. Accessories may be fabric flowers, charms, gems, decorative magnet attachments and other decorative attachments or therapeutic magnets. Decorative attachments can further be adhered directly on the scrunchie.

It is an object of the present invention to provide a scrunchie that its ruffle distribution can be easily adjusted by the user.

It is another of the present invention to provide a hair accessory with attachments to attach various accessories in a sturdy and stable manner and prevent diminishing effect of the desired designs on the accessory.

It is another object of the present invention to provide a wearing accessory, which can be used as a hair accessory to hold hair or encircled about the wrists as a therapeutic wrist-band.

It is another object of the present invention to provide a wearing accessory in a kit with various decorative accessories and corresponding attachments to use as a hobby for collecting and trading accessories.

It is another object of the present invention to provide a method for adjusting the length of the wearing accessory to reach to a desired length by pulling the elastic band while fastening it on the fabric tube to create a desired length.

These and other objects and advantages of the invention will become apparent upon reference to the accompanying drawings and the following detailed description.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1A is a perspective view of the wearing accessory of the present invention being worn as a hair fastener;

FIG. 1B is a perspective view of the present invention showing the accessory being worn on the wrist as a wrist band;

FIG. 2 is a perspective view of the fabric tube member of the preferred embodiment of the invention;

FIG. 3 is a plan view of the fabric tube to show how the tube is prepared prior to use in the present invention;

FIG. 4 is a plan view of the elastic band used in the present invention to show how the elastic band is prepared prior to installment into the fabric tube;

FIG. 5A is a cross-sectional view showing the attachments according to the present invention;

FIG. 5B is a perspective view showing the attachments according to FIG. 5A;

FIG. 6A is a perspective view showing the insertion of the elastic band into the slits;

FIG. 6B is a perspective view of the final shape of the present invention to receive decorative accessories;

FIG. 7 is a plan view of another embodiment of the present invention to show how the fabric tube is prepared prior to use;

FIG. 8 is a perspective view of the fabric tube according to FIG. 7;

FIG. 9 is an isometric view of the present invention according to FIG. 7;

FIG. 10 is an isometric view showing insertion of the elastic band of FIG. 4 in the slits according to FIG. 7;

FIG. 11 is an isometric view of the annular fabric tube after assembly with elastic band according to FIG. 7;

FIG. 12 is a perspective view of another embodiment of the present invention:

FIG. 13 is a cross-sectional view showing the attachments according to FIG. 12;

FIG. 14 is an isometric view of the annular fabric tube after assembly with elastic band according to FIG. 12, and

FIG. 15 is a perspective view of various attachments and accessories used according to the principals of the present invention.

### DETAILED DESCRIPTION OF PREFERRED EMBODIMENTS

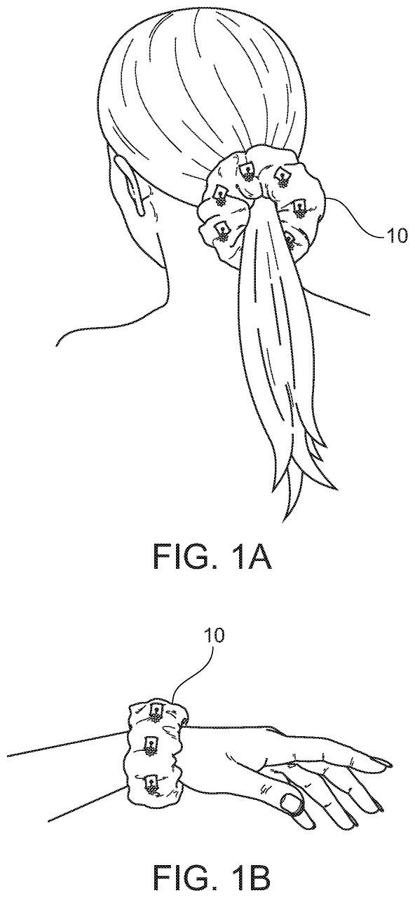

According to FIGS. 1A and 1B the present invention 10 is a hair accessory or a scrunchie that may be twisted and doubled over to hold hair or encircled about the wrists as a decorative or a therapeutic wrist-band.

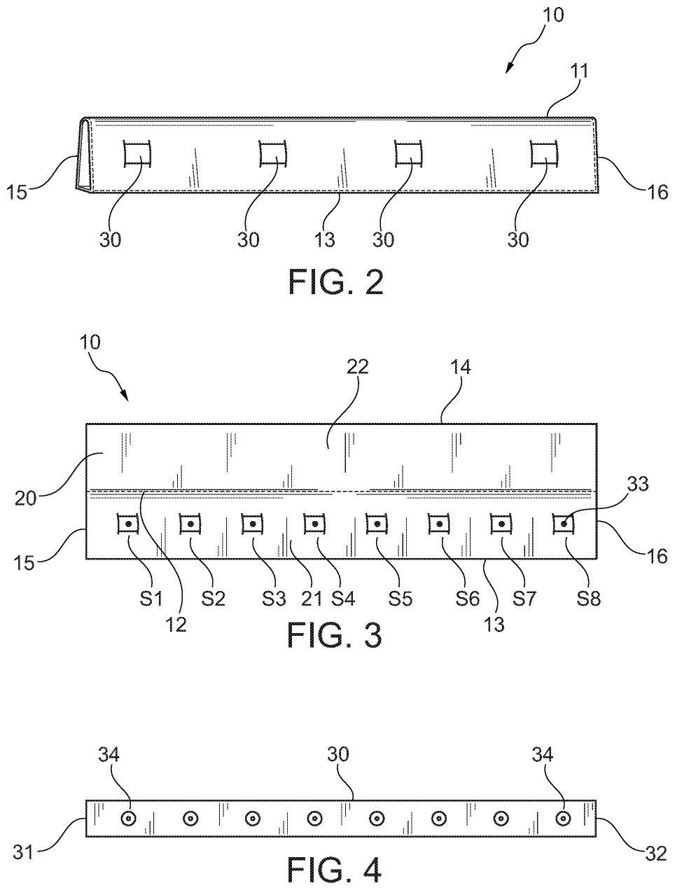

In general, and with reference to FIGS. 2 to 6B the accessory 10 includes an annular tube 11 of fabric and an elastic band 30. To better understand the construction and features of the accessory 10, the method of construction of the present invention 10 is described initially. According to FIG. 3 the tube 11 is shown prior to its assembly with the elastic band 30. The tube 11 may be made of various fabric materials such as, but not limited to, jersey, lycra, cotton knit, interlock, and thermal materials, and of natural or synthetic fibers. The tube 11 is formed from a rectangular piece of fabric 20 that is folded and sewn into the tube. The tube 11 is then formed in an annulus in a well-known manner and, as a result, has a longitudinal seam 13 along one outer edge.

Although the invention is not limited to particular dimensions, it is believed useful to provide an example of dimensions for preferred embodiments to show how the invention is accomplished. Accordingly, the circumference of the tube, from end 15 to end 16, is preferably from about 20 to 24 inches, and the width of the tube, from edge 13 to edge 14, is preferably from about 2 to 5 inches.

The assembly of the tube is shown in FIG. 3 performing the steps of construction of the present invention. The tube 11 is formed of a rectangular fabric 20 with a length of about 25 inches and a width of about 10 inches, which will be folded longitudinally from the center of the fabric 12 and stitched together, resulting in a fabric tube 11. The fabric has a first portion 21 and a second portion 22. The ends of the tube 15 and 16 are stitched together to constitute a tubular annulus. The present invention 10 provides a plurality of slits placed adjacent to each other on longitudinal surface of the tube, which will be disclosed in detail.

In accordance with the first embodiment of the present invention 10, the first step of the method is to cut pairs of slits S1 to S8 on the first portion of the fabric 21. Each pair of slits S1 to S8 comprises of two cut-outs with a length of about ½ inch and spacing of about ½ inch. The elastic band 30 is passed in and out of the slits, and it sticks out of the small spacing between each pair of slits: out from S1a and in from the second cut-out S1b. The elastic band extends inside the tube to reach to the next pair of slits S2. This process will be continued until the elastic band 30 reach the last slit portion S8. It is to be understood the example of the number of slit portions and dimensions for preferred embodiments is to show how the invention works and not to limit the number and sizes of the slits and their spacings.

As shown in FIG. 4 the elastic band 30 is formed from a flat strip, preferably of cotton elastic, having opposite ends 31 and 32 with a dimension of about 10 inches long, end-to-end, and a width of about ½ inches. It is thus understood that the elastic band 30 has a length in its contracted condition less than the circumference of the tube 11 that is, less than about one-third to less than about one-half, thereby forms ruffles on circumference of the tube. Since the user has access to the elastic band, the ruffle distribution can be adjusted by moving the fabric over the elastic band. For instance, in order to make the scrunchie comfortable behind the neck of the user, the ruffles can be removed from the side of the scrunchie that sits on the neck.

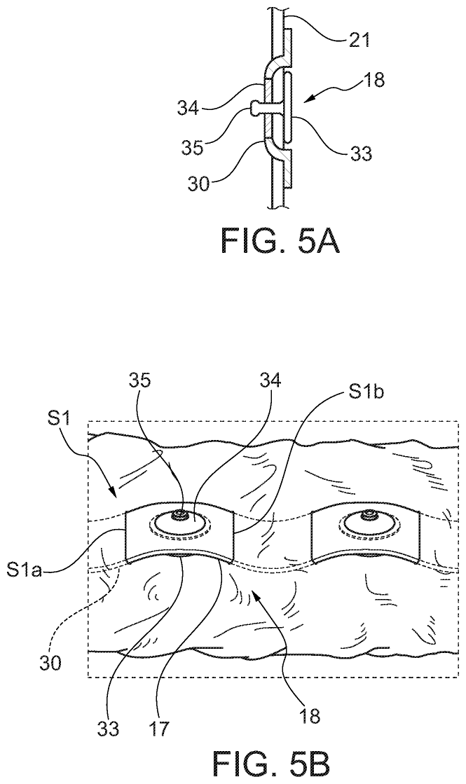

The present invention 10 provides attaching means to attach accessories on the fabric. FIGS. 5A and 5B show one slit portion with the detail parts of the attaching means 18. The attaching means 18 comprises a stud element 33 attached on the fabric and a socket element 34 attached on the elastic band to fasten the elastic band thereon. The socket elements 34 are attached on the length of the elastic band 30 in a manner, according to the example embodiment depicted in FIG. 4, in which eight socket elements 34 in an equal distance apart to each other are shown. The first and the last socket elements are attached in a specified distance from the distal end 31 and proximal end 32, allowing tying the ends.

The socket element of the elastic band 34 is engaged to the stud member 33 attached to the slit portion of the fabric. The stud member 33 is attached to the center of the fabric 17 in between the first cut-out S1a and second cut-out S1b of the slit pair to secure the elastic band 30 thereon. The protruded portion of the stud member has a distal end 35, which will further receive various attachments to receive decorative accessories.

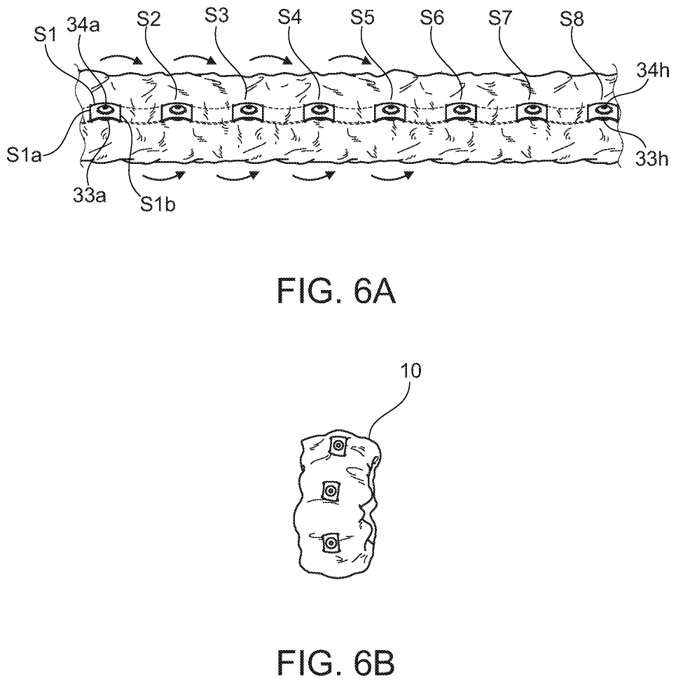

According to FIGS. 6A and 6B the next step is to move the elastic band 30 in an arrangement from its distal end 32 into the slits of the fabric beginning from S1 in a manner that it sticks out of S1a, then pulling the elastic band out and then inserting the elastic band into S1b to reach to the next pair of slits S2. The elastic band 30 goes in and out of all pairs of slits. The elastic band 30 is then pulled so that the fabric is gathered into undulations thereby cause the tube to create ruffles. The next step is to fasten the first socket element of the elastic band 34a on the first stud of the fabric 33a and last socket element 34h on the last stud element 33h and pull the elastic band 30 to fasten each socket element 34 to the corresponding stud element 33. The process will continue to the last slit S8 until the last socket element of the elastic band 34 is fastened to the last stud element 33.

The next step is to fold the first portion 21 longitudinally from the center of the fabric 12 and align the edges 13, 14 and stitch together, resulting a fabric tube 11. The tube 11 is then formed in an annulus in a well-known manner. The distal end and proximal end of the elastic band 30 is then tied together for more stability. The next step is to sew the ends of the tube 15 and 16 together to constitute a tubular annulus. This forms the final shape. Typically, the circumference of the tube from one end 15 to the other end 16 is about 20 inches and its width from edge 12 to edge 13 is about five inches. The elastic band goes all the way around the tube and contracts.

The edges 13 and 14 can also first be stitched together, resulting a fabric tube 11 and then the elastic band 30 is inserted all the way around the tube in the same method. Any number of stud elements 33 may be attached to the fabric, these stud elements 33 may be evenly spaced as shown or spaced at varying distances from one another.

The user is able to customize the tube 11 by attaching decorative accessories in a sturdy and stable manner in a predefined arrangement and fastens hair in a more efficient and comfortable manner, especially with decorative accessories. The method of construction of the present invention 10 further permits a user to adjust the length of the wearing accessory to reach to a desired length. The elastic band can be pulled so that to cross one or more slits to provide more ruffles and shorten the length of the hair accessory. This will result in a length which does not require additional twisting around the hair that creates large knot of twisted fabric. Fabric knot in the scrunchie can decrease comfort and diminish desired decorations.

The present wearing accessory permits the user to both easily place his or her ponytail through the opening of the wearing accessory while at the same time securing the decorative accessory in a sturdy and neat arrangement thereon. Secured attachments are especially needed when a user has rapid movements, such as playing a sport where rapid movement of the head in different directions can cause the decorative attachment to loosen and fall.

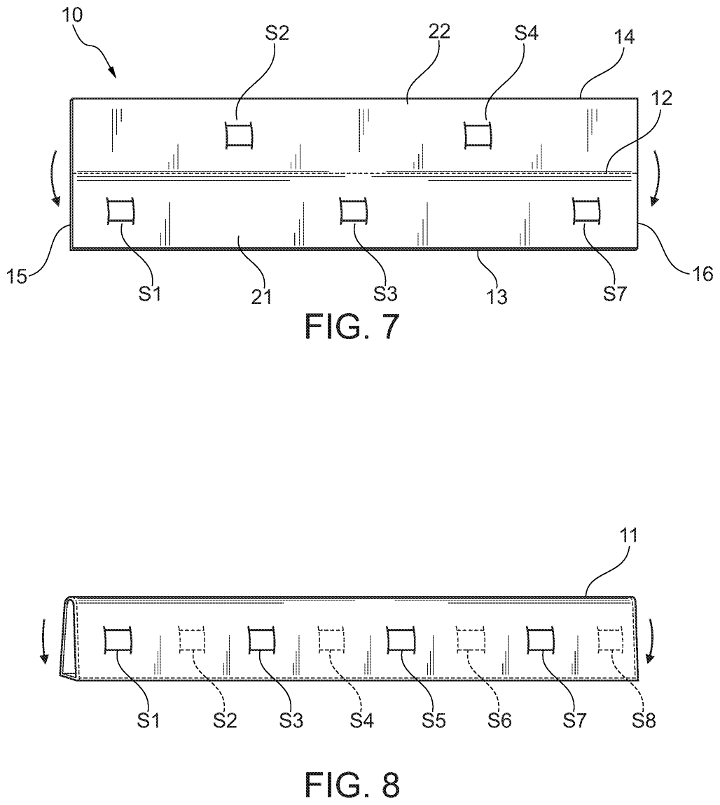

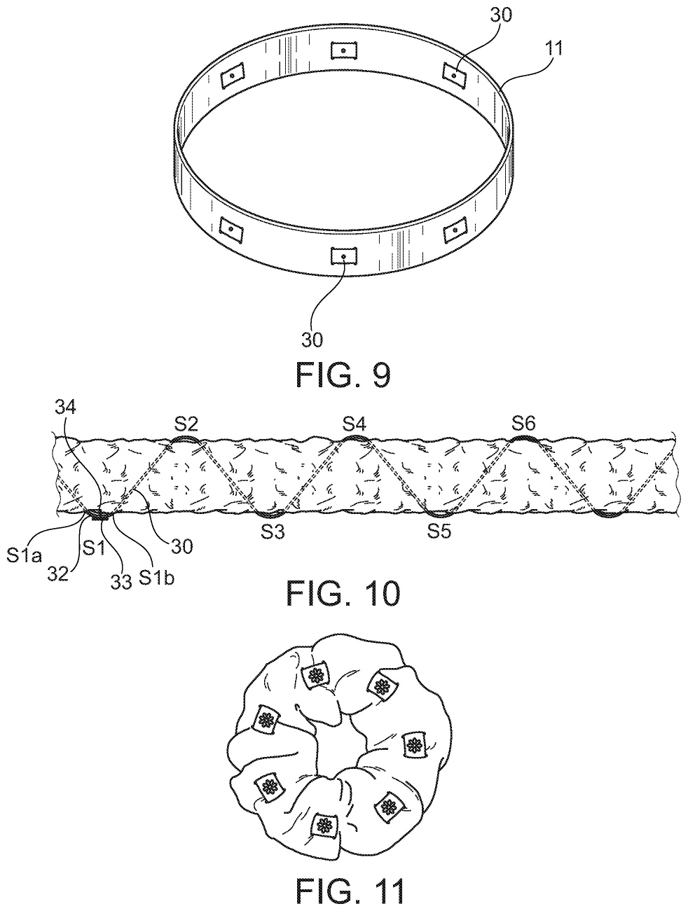

In another embodiment of the present invention, as shown in FIGS. 7 to 11, the pairs of slits are arranged on both first portion 21 and second portion 22. Slit pairs S1, S3, S5 and S7 on the first portion 21 and slit pairs S2, S4, S6 and S8 on the second portion 22 are arranged in a manner so that when the fabric is folded the slits of the second portion 22 will position in between the slits of the first portion 21 in one row. To achieve the principals of the present invention in this embodiment, the first portion 21 is folded longitudinally from the center of the fabric 12 and the aligned edges 13 and 14 are stitched together, resulting a fabric tube 11.

The next step is to move the elastic band 30 in an arrangement as shown in FIG. 10. The elastic band is inserted in and out of the first pair of slits S1 to the last pair of slits S8. The distal end 32 into the slits of the fabric beginning from S1 in a manner to stick out of S1a, pull the elastic band out and fasten the first socket of the elastic band 34 on first stud of the fabric 33 and then stick the elastic band into S1b and pull the elastic band to reach to the next slit S2. The process will continue to the last slit S8 until the last socket of the elastic band 34 is fastened to the last stud 33 of the last slit S8 to accomplish the final shape. In this embodiment both sides of the tube 11 provide decorative attachment portions.

In another design of this embodiment, the first portion 21 is made from a first piece of fabric having a first design and a second portion 22 is made from a second piece of fabric having a second design, which can be decorated with decorative accessories. In this case, two pieces of fabric are joined together to form inner seam 12 by sewing or other means to create a joined piece of fabric.

The distal end and proximal end of the elastic band is then tied together and stick into the tube and the ends of the tube 15 and16 stich together to constitute a tubular annulus. In this embodiment either side of the accessory can be displayed equally well.

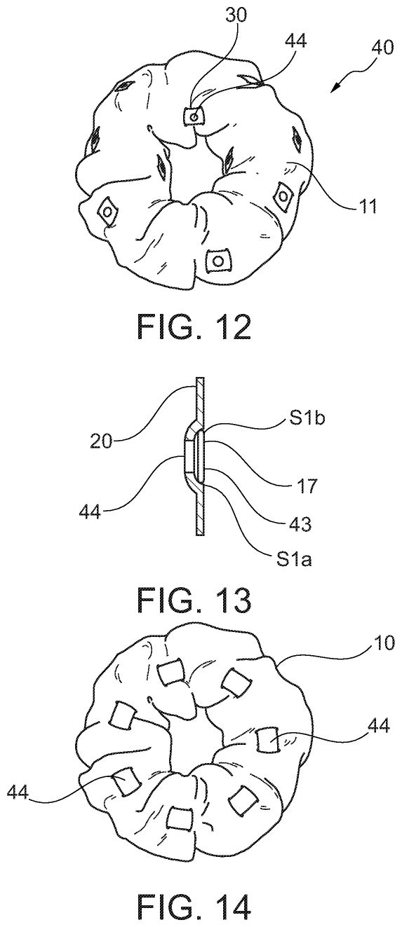

In another embodiment 40, According to FIGS. 12 to 14, the present invention provides magnet attachments to attach accessories on the fabric. The attaching member 44 is a double-sided magnet snap fastener attached on the length of the elastic band 30 in a manner. The figure shows eight double sided magnet fasteners 44 in an equal distance with each other. The magnet fastener 44 is attachable to the stud member 43 fastened to the slit portion 17 of the fabric. The stud member 43 can also be a magnet stud 43 attached to the center of the fabric 17 in between the first cut-out S1a and second cut-out S1b of the slit portion S1 to secure the elastic band 30 thereon and receive a decorative accessory with magnet attachment. The double-sided magnet fastener 44 receive decorative accessories with magnetic snap fasteners or a magnet cap.

This embodiment of the invention 40 can also be used as a therapeutic magnetic wrist-band. It is believed that use of the magnetic wrist bands promotes rapid recovery of persons suffering from arm pump-up. They are used by many athletes, tennis players, golfers and also elderlies.

A suitable therapeutic magnet such as earth magnet is attached to each double-sided magnet fastener 44 on elastic band 30. Earth magnets which can be used for example nickel-plated neodymium iron boron magnets in a shape to be attached on elastic band 30. The wrist band 40 is circled around the wrist in a manner to position the magnets to face wrist of the body. In particular, the strength of the magnets and the number can be altered or varied to suit the needs of each application. The tube 10 can be used from both sides as a wrist band 40 so that the user can design a combination of magnet and decorative accessories on the tube 10.

In another embodiment the elastic band 30, the elastic bands is made of compatible synthetic material having an active surface with permanent magnetic particles embedded therein. The elastic band is covered of a magnetic therapeutic layer of magnetic powder applied directly to at least one side of the elastic band. The elastic band 30 can be then inserted into the tube 11.

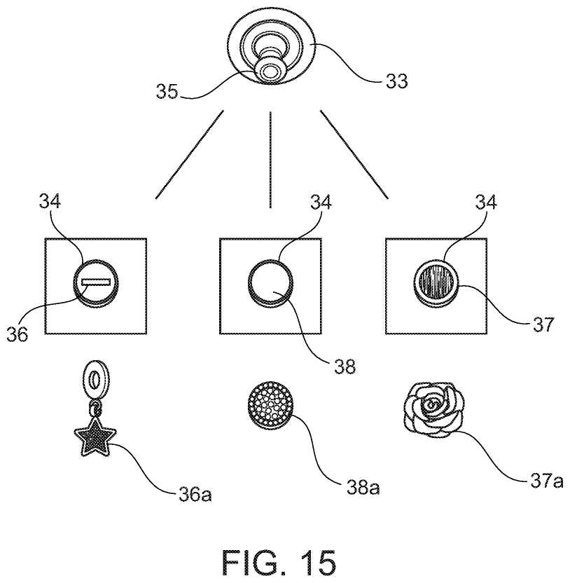

In a preferred embodiment, the present invention may be produced as a kit with various decorative accessories with corresponding attachments. According to FIG. 15 various shapes of attachments can be provided on the distal end of the stud member 35 of the fabric to attach decorative accessories. The socket member of the elastic band may also provide various attachments on its rear side to receive decorative accessories. The kit for hair band with interchangeable attachments comprising:
at least one hair band;
various attachments on the rear side of the socket members fixedly to receive decorative accessories;
a plurality of decorative accessories each having corresponding clip attachments;
a plurality of decorative accessories each being glued to the hair band;
a plurality of decorative accessories each being sewed to the hair band;
a plurality of decorative accessory each having corresponding Velcro attachments;
a plurality of decorative attachments each having corresponding magnet attachments, and
a plurality of therapeutic magnet attachment each having corresponding magnet attachments. Attachments may be clip fasteners 36, Velcro attachments 37, magnet attachments 38, hook and loop type fasteners, glue and the like with corresponding decorative accessories.

It is again emphasized that neither the accessory 10 nor the method is limited to the particular dimensions as described above and maybe of larger or smaller sizes while still incorporating the basic principles of the invention.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1) A hair band with interchangeable attachments comprising:
a tube formed into a circular shape from a fabric having a rectangular shape, said fabric has a pair of outer edges, a pair of end edges, and a fold line, said fabric is divided into a first wall portion and a second wall portion on opposite sides of said fold line, wherein said fabric is folded in half lengthwise along the fold line and said pair of outer edges are stitched together to form a lengthwise seam, and thereby forming said tube, and said pair of end edges are joined forming a transverse seam in said tube, said tube having a circumference with a predetermined length, an inner portion and an outer portion;
an elastic band having a distal end and a proximal end and a length shorter than said tube in its unstressed position and slightly equal length in stressed position, thereby gathering the tube into a contracted and ruffled condition, and
a plurality of slits on said first wall portion of said fabric spaced substantially in parallel relation to said pair of outer edges, wherein said plurality of slits comprising a set of vertical cut-outs perpendicularly provided apart each other longitudinally on said tube whereby said elastic band runs inwardly and outwardly in an interlacing manner into and out of each adjacent slits and extend inside said tube, thereby gathering said tube into a contracted and ruffled condition.

2) The hair band of claim 1, wherein said plurality of slits are provided on said first all portion and said second wall portion spaced substantially in parallel to relation to said outer edges so that said elastic band being interlacing inserted inwardly and outwardly into said slits and extending through inner portion and outer portion of said tube, causing said tube to be undulated circumferentially.

3) The hair band of claim 1, further having a plurality of securing attachments to secure said elastic band on said tube.

4) The hair band of claim 1, further having a plurality of interchangeable accessories attached to said plurality of securing attachments.

5) The hair band of claim 3, wherein each of securing attachments comprising a stud element and a socket element.

6) The hair band of claim 3, wherein said plurality of securing attachments comprising:
a plurality of stud elements secured to said inner portion in location between each slit in an equal distance apart between opposite end edges lengthwise such that each stud element is positioned between slit members, and
a plurality of socket elements attached in a distance apart between said distal end and proximal end of said elastic band attachable to said stud elements, each socket element having front and back sides, each of said back sides including corresponding attachments positioned at opposite ends thereof and attachable to said attachments of said interchangeable accessories.

7) The hair band of claim 5, wherein said stud element comprising a protrusion extending outward from said inner side of said tube through the outer side to receive said plurality attaching accessories.

8) The hair band of claim 4, wherein said plurality interchangeable accessories having a plurality of corresponding attachments to be secured on said socket elements.

9) The hair band of claim 3, wherein said plurality of securing attachments, comprising a double-sided magnet socket attached on the length of said elastic band in an equal distance to each other attachable to said stud member on said tube to receive decorative accessories with magnetic snap fasteners or a magnet caps.

10) The hair band of claim 3, wherein said plurality of securing attachments comprising one of hook and loop attachments being a double-sided socket member having a loop member to receive a hook or snap fastener of a decorative accessory.

11) The hair band of claim 3, wherein said plurality of securing attachments comprising one of a hook and loop attachment comprising a double-sided socket member having a Velcro member to receive a decorative accessory with Velcro attachment.

12) The hairband of claim 1, wherein the fabric is selected from the group consisting of silk, rayon, organdy, linen, cassimere, poplin or other suitable material.

13) The hair band of claim 1, wherein the first wall is made from a first fabric and the second wall is made from second fabric.

14) A hair band with interchangeable attachments and useable as a therapeutic wristband, comprising:
an annular tube formed into a circular shape from a rectangular piece of fabric, said fabric has opposite outer edges, opposite end edges and a fold line, said fabric is divided into a first wall portion and a second wall portion on opposite sides of said fold line, wherein said fabric is folded in half lengthwise of the fold line and said outer edges are stitched together to form a lengthwise seam whereby forming a tube and said end edges are joined forming a transverse seam in said tube, said tube having a circumference of predetermined length having an inner portion and an outer portion;
an elastic band having a distal end and a proximal end and a length shorter than said tube in its stressed position and slightly equal length in unstressed position;
a plurality of slit portions on said first wall portion an said second wall portion of fabric spaced substantially in parallel relation to said outer edges, wherein said slit portions comprising a set of vertical cut-outs perpendicularly provided apart each other longitudinally on said tube whereby said elastic band run inwardly and outwardly in an interlacing manner into and out of each said adjacent slit portion and extend inside said tube, thereby gathering said tube into a contracted condition;
a plurality of securing attachments to secure said elastic band on said tube;
a plurality of therapeutic magnet attachments, and -
plurality of decorative attachments.

15) The wrist band of claim 14, wherein said attaching means comprising:
a plurality of stud elements secured to said inner portion of said tube in location between each slit of slit portion in an equal distance apart between opposite end edges lengthwise such that each stud element is positioned between slit members;
a plurality of magnetic socket elements attached in a distance apart between said distal end and proximal end of said elastic band attachable to said stud elements;
whereby said magnetic socket elements are engaged to said stud members, said plurality of therapeutic magnet attachments are attached on said magnetic socket elements on said first wall facing the wrist and said plurality of decorative accessory are attached on said second wall.

16) The wrist band of claim 14, wherein said attachments, comprising a double-sided magnet socket attached on the length of said elastic band in an equal distance to each other attachable to said stud member on said tube to receive a therapeutic magnet,
whereby said wrist band is circled around the wrist in a manner to position said magnets to face wrist of the body.

17) The wrist band of claim 14, wherein said therapeutic magnets are selected from the group consisting of earth magnets such as nickel-plated neodymium iron boron magnets.

18) The wrist band of claim 14, wherein said elastic band is composed of compatible synthetic material having an active surface with permanent magnetic particles embedded therein or is covered of a magnetic therapeutic layer of magnetic powder applied directly to at least one side of the elastic band.

19) A method of making a hair band with interchangeable attachments, said hair band being made from a rectangular fabric having opposite outer edges, opposite end edges and a fold line, said fabric is divided into a first wall portion and a second wall portion on opposite sides of said fold line, an elastic band having a distal end and a proximal end and a length shorter than said tube in its stressed position and slightly equal length in unstressed position, said method comprising:
folding said fabric in half lengthwise of the fold line;
stitching together said outer edges to form a lengthwise seam thereby forming a tube;
cutting a set of vertical cut-outs perpendicularly apart each other creating a plurality of slit portions on said wall portions;
fastening a plurality of stud elements to said inner portion;
providing a plurality of socket elements on said elastic band in a distance apart between said distal end and proximal end attachable to said stud elements;
sliding the elastic band inwardly and outwardly from its distal end into said cut-outs beginning from first slit and pulling said elastic band out and fastening the first socket of said elastic band on first stud of said tube;
sticking said elastic band inwardly through said adjacent slit and pulling said elastic band to reach to the next slit thereby gathering and creating ruffles;
continuing to fasten each socket element to the corresponding stud element until the last socket is fastened to the last stud;
knotting said distal end and proximal end, and
stitching said end edges together forming into a circular shape, and attaching a plurality of selected accessory having various corresponding attachments on said socket elements.

20) The method of claim 19, wherein said elastic band is adjustable to reach to a desired length by pulling said elastic band while fastening it on said fabric tube by sliding said elastic band inwardly and outwardly from its distal end into said cut-outs beginning from first slit and pulling said elastic band out and fastening said first socket of said elastic band on first stud of said tube and sticking said elastic band inwardly through said adjacent slit and pulling said elastic band to reach to the desired slit thereby gathering and creating ruffles and adjust the length and fasten each socket element to the corresponding stud and knotting said distal end and proximal end of the elastic band.
